(19) United States
(12) Patent Application Publication
(10) Pub. No.: US 2026/0048005 A1
(43) Pub. Date: Feb. 19, 2026

(54) INJECTABLE THERMOSENSITIVE HYDROGELS FOR A SUSTAINED RELEASE OF IRON NANOCHELATORS

(71) Applicant: The General Hospital Corporation, Boston, MA (US)

(72) Inventors: Hak Soo Choi, Needham, MA (US); Homan Kang, East Boston, MA (US)

(21) Appl. No.: 19/104,053

(22) PCT Filed: Aug. 11, 2023
(86) PCT No.: PCT/US2023/030074

§ 371 Date: Feb. 14, 2025

Related U.S. Application Data
(60) Provisional application No. 63/371,526, filed on Aug. 16, 2022.

(51) Int. Cl. A61K 9/00; A61K 31/164; A61K 47/34; A61K 47/36; A61P 39/04

(52) U.S. Cl. CPC A61K 9/0024; A61K 31/164; A61K 47/34; A61K 47/36; A61P 39/04

(57) ABSTRACT

Disclosed herein are injectable hydrogel formulations prepared by integrating crosslinked hyaluronic acid into Pluronic F127 for an extended release of DFO nanochelators. Methods of manufacture and of use are also provided.

### CLAIM OF PRIORITY

This application claims the benefit of U.S. Patent Application Ser. No. 63/371,526 filed on Aug. 16, 2022. The entire contents of the foregoing is hereby incorporated by reference.

### FIELD

This disclosure relates injectable hydrogel formulations prepared by integrating crosslinked hyaluronic acid into Pluronic F127 for an extended release of DFO nanochelators and methods of manufacture and of use thereof.

### BACKGROUND OF THE INVENTION

Iron is an essential metal yet high iron stores are toxic due to metal-induced oxidative stress that promotes organ damage including heart failure, liver cirrhosis, diabetes, and neurodegenerative diseases. Primary iron overload (hereditary hemochromatosis) is one of the most common genetic diseases, affecting 1 million people worldwide, mainly the Caucasian population. Secondary iron overload can occur in patients with hemoglobinopathies, such as thalassemia major, sickle cell anemia, aplastic anemia, myelofibrosis, myelodysplastic syndrome, and Diamond-Blackfan anemia because they require chronic blood transfusions. Since there is no recognized active pathway of iron excretion, chelation therapy has been widely used to improve disease conditions in patients with iron overload, especially transfusion-associated iron accumulation.

There are three small molecule-based iron chelators currently in use: deferoxamine (DFO), deferiprone (DFP), and deferasirox (DFX). In particular, DFO has shown good therapeutic efficacy since being approved by the U.S. FDA in 1968. However, the very short half-life (e.g. 5-15 min in rodents) of DFO requires repeated injections or continuous infusions which considerably reduce patient compliance and their quality of life. The other two chelators, DFP and DFX, are orally administrable and therefore overcome this compliance issue. However, DFP and DFX cause significant adverse effects including gastrointestinal tract bleeding, agranulocytosis, neutropenia, thrombocytopenia, hepatic fibrosis, and kidney failure. Although other orally active iron chelators, like derivatives of desferrithiocin, are in clinical trials or preclinical phases, they have nephrotoxic effects.^{[8]}

Currently, several sustained drug release systems have been developed such as hydrogels, patches, implantable drug devices, and infusion pumps, which significantly improve the compliance and adhesion issues of patients. Especially, injectable thermosensitive hydrogels have been frequently used for drug delivery because they do not require additional chemical reactions and external stresses (e.g., light or pressure) to crosslink. However, many polymeric hydrogels show potential toxicity. Thus, it is necessary to develop a hydrogel formulation with FDA-approved materials to accelerate the progress of clinical trials.

### SUMMARY

The present disclosure relates to injectable thermosensitive hydrogels for the long-term release of DFO-NPs for iron chelation therapy.

In some embodiments, the disclosure provides a sustained release, injectable composition comprising an iron chelation agent and at least one hydrogel.

In some embodiments, the disclosure provides a sustained release, injectable composition for iron chelation therapy in a human patient comprising an iron chelation agent and a hydrogel, said composition capable of release over a two week period. In some embodiments, the disclosure provides a sustained release, injectable composition for iron chelation therapy in a human patient comprising an iron chelation agent and a hydrogel, said composition is released over a two week period.

In some embodiments, the disclosure provides a method of treating iron overload in a patient in need thereof comprising administering a therapeutically effective amount of a sustained release, injectable composition comprising an iron chelation agent and at least one hydrogel to the patient.

In some embodiments, the disclosure provides a method of reducing the amount of free metal ions in a cell or tissue sample, comprising contacting a cell sample or a tissue sample with a therapeutically effective amount of a sustained release, injectable composition comprising an iron chelation agent and at least one hydrogel.

In some embodiments, the disclosure provides a method of treating a disease associated with an abnormal amount of free metal ions in a subject, comprising administering to a subject determined to have an abnormal level of free metal ions a therapeutically effective amount of a sustained release, injectable composition comprising an iron chelation agent and at least one hydrogel.

In some embodiments, the disclosure provides a method of preparing a sustained-release, injectable composition useful iron chelation therapy comprising the steps:
a) cross-linking HA;
b) making the cross-linked HA into particles;
c) adding the particles of cross-linked HA into a solution of DFO-NP; and
d) further adding F127 to the resulting solution of step c).

Unless otherwise defined, all technical and scientific terms used herein have the same meaning as commonly understood by one of ordinary skill in the art to which the present application belongs. Methods and materials are described herein for use in the present application: other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF THE DRAWINGS

FIG. 1. Schematic diagram of DFO-NP loaded injectable hydrogels. DFO-NPs were loaded in crosslinked-hyaluronic acid (xHA), and F127 was formulated as an outer hydrogel. Sustained-release of DFO-NPs from the hydrogel significantly improves their pharmacokinetics and minimized off-target tissue distribution.

FIG. 2. ¹H-NMR spectra of DFO, ZW-EPL⁺, ZW-EPL⁻, and DFO-NP. DFO to ZW-EPL⁻ ratio was calculated by peak integration of the ZW-EPL⁻ protons (positions α′ at 4.12 ppm), and DFO protons (position e,l,s at 3.6 ppm). The calculated values of DFO per chain were based on the assumption of 30 lysine units per entire ZW-EPL⁻.

FIG. 3. Hydrodynamic diameter (HD) analysis of DFO-NPs. A) Size-exclusive chromatography measured at 254 nm (black line) and 760 nm (red line), respectively. Arrowheads and numbers indicate the corresponding HD of standard proteins: Aprotinin, 1.96 nm: ribonuclease, 3.28 nm: ovalbumin, 6.10 nm: thyroglobulin, 17.00 nm. B) The standard calibration curve of HD using the standard proteins.

FIG. 4. Optophysical properties of DFO-NPs. A) Optical properties including absorption and fluorescence spectra at a concentration of 100 μM. B) UV-vis absorption spectra of Fe(III)-titrated DFO-NP (blue line indicates DFO-NP solution before adding iron solution, red lines indicate after titration after titration with the iron solution. C) A titration curve of absorbance change (A−A₀).

FIG. 5. In vitro cellular uptake and viability tests for DFO-NPs. A) Cellular uptake study for ZW-EPL⁺ (5 μM) and DFO-NP (5 μM) on NIH3T3 and H23 cells. B) Cell viability of NIH3T3 cell at various concentrations of DFO-NP.

FIG. 6. Rheological properties of hydrogel formulations. a) crosslinked hyaluronic acid (HA: 7%) and b) DFO-NP loaded HA (DFO-NP/HA; 30/7%) c) Pluronic F127 (F127; 30%) and d) DFO-NP loaded F127 (DFO-NP/F127; 30/30%), e) HA and F127 integrated hydrogel (HA/F127 7/30%), and f) DFO-NP loaded HA/F127 (DFO-NP/HA/F127; 30/7/30%).

FIG. 7. In vivo longitudinal monitoring of DFO-NP release from various hydrogel formulations. A) Representative color and NIR fluorescence images of CD-1 mice subcutaneously injected with DFO-NP and DFO-NP loaded hydrogels, such as DFO-NP/F127, DFO-NP/HA, and DFO-NP/HA/F127 for up to 14 d post-injection. White arrowheads indicate remaining hydrogels on day 14. Exposure time: 50 ms. B) Longitudinal profiling of signal-to-background ratio (SBR) of hydrogel injected sites (region of interest; ROI) against background signal (Bg) (n=3 per group, mean #s.e.m.). C) SBR of skin and hydrogels against muscle after peeling off the skin at 14 d. p values<0.05 were considered significant: *p<0.05, ****p<0.001.

FIG. 8. Biodistribution of DFO-NPs at 14 d post-injection. A) Color and NIR fluorescence images of abdomen, chest, and resected organs of mice subcutaneously injected with DFO-NP and DFO-NP loaded hydrogel formulations. B) Signal-to-background ratio (SBR) of resected organs and hydrogel against muscle. He, heart; Lu, lung; Li, liver; Pa/Sp, pancreas/spleen; Ki, kidney; Du, duodenum; In, intestine; Mu, muscle.

FIG. 9. Pharmacokinetic analysis for DFO-NP and DFO-NP/F127, DFO-NP/HA, and DFO-NP/HA/F127 hydrogels. A) Plasma concentration decay curve, and B) area under the curve (AUC).

FIG. 10. H&E staining images (10×) of heart, lung, liver, spleen, and kidney in saline and DFO-NP/HA/F127 groups.

FIG. 11. Representative fluorescence images of blood samples in capillary tubes at each time point.

FIG. 12. In vivo toxicity test in mice injected with saline and DFO-NP/HA/F127 hydrogel. A) H&E staining images (20×) of heart, lung, liver, spleen, and kidney in each group. B) Serum aspartate transferase (AST), alanine transferase (ALT), and AST/ALT ratio. C) Blood urea nitrogen (BUN) and creatinine (CREA). Mice were sacrificed on 14 d post-injection for analyses. p values<0.05 were considered significant: *p<0.05.

FIG. 13. Therapeutic efficacy of DFO-NP loaded hydrogel formulations in animal model. Male CD-1 mice were fed with 1% carbonyl iron diet for 1 week and then treated with a single subcutaneous injection of saline, blank NP/HA/F127 (84 μmol/kg as NP), or DFO-NP/HA/F127 (84 μmol/kg as NP). After dosing, mice were fed facility regular chow. Organs were collected 3 weeks after drug administration (n=3, mean±SEM). p values<0.05 were considered significant: *p<0.05.

### DETAILED DESCRIPTION

### Compositions

In some embodiments, the disclosure provides a sustained release, injectable composition comprising an iron chelation agent and at least one hydrogel.

In some embodiments, the iron chelation agent is defereoxamine (DFO), which has the formula:

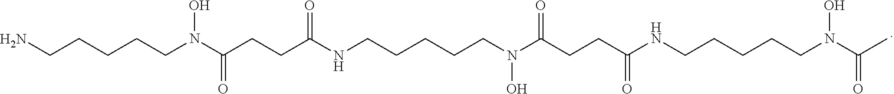 [SMILES: CC(=O)N(O)CCCCCNC(=O)CCC(=O)N(O)CCCCCNC(=O)CCC(=O)N(O)CCCCCN]

In some embodiments, the iron chelation agent is a pharmaceutical salt of DFO, such as the mesylate salt. In some embodiments, the DFO is DFO nanochelator (DFO-NP).

In some embodiments, DFO-NP is a compound of formula A-B. A is a group comprising 1, 2, 3, or 4 anionic groups which are each sulfonate. In some embodiments, A comprises 1 or 2 anionic groups which are each sulfonate. In some embodiments, A is selected from the group consisting of formulas A-1, A-2, A-3, A-4, A-5, and A-6:

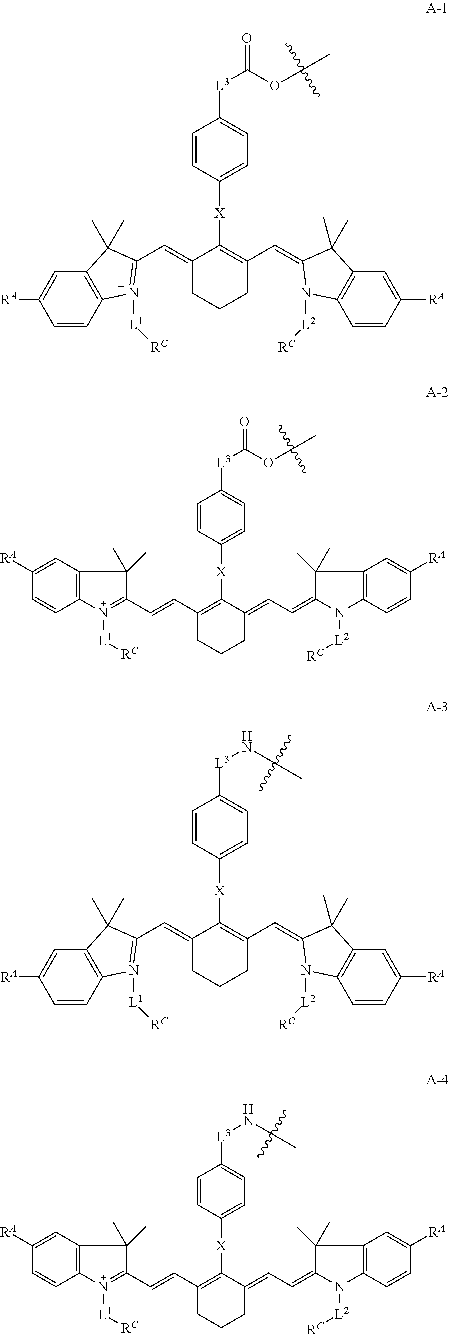

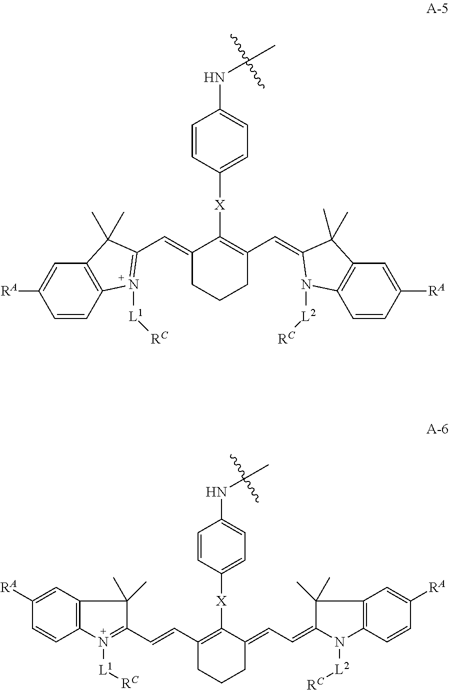

wherein:
[symbol] indicates the bond between A and B;
X is selected from the group consisting of a bond, CH₂, NH, —NH—C₁₋₆alkylene-, O, and S; each
R^{A} is an independently selected anionic group; each R^{C} is an independently selected cationic group; and L¹, L², and L′ are each an independently selected C₁₋₆ alkylene group.
   In some embodiments A is selected from of:

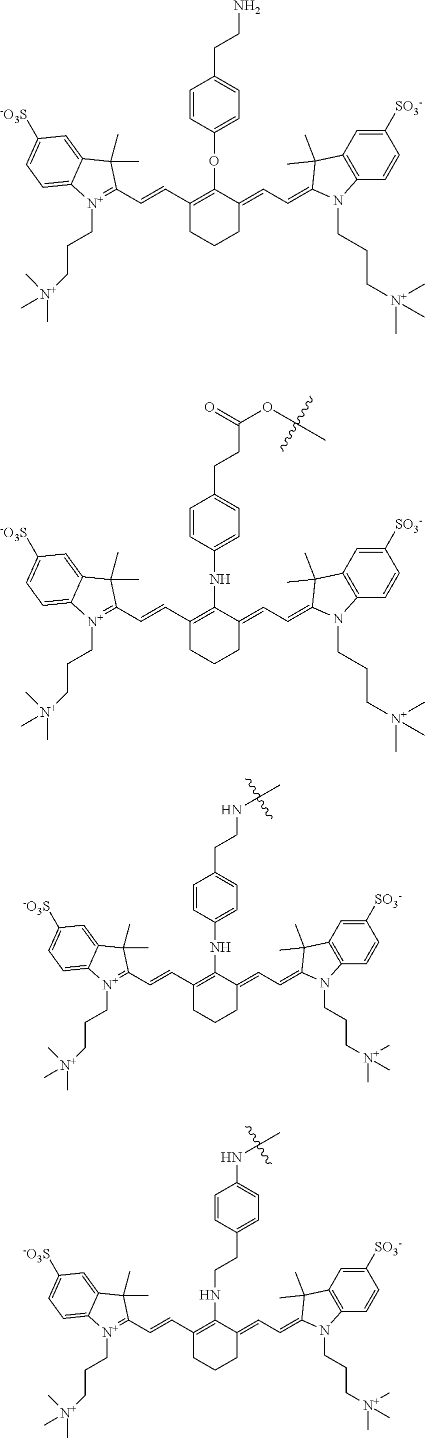

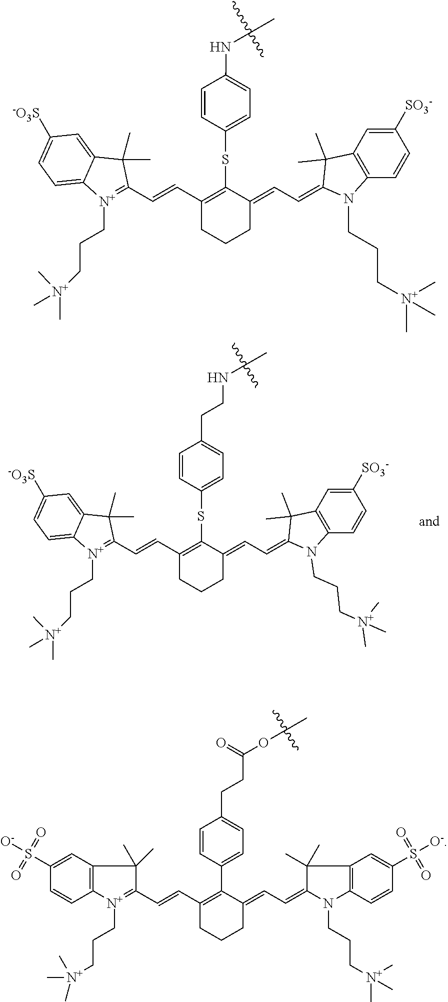

In some embodiments A is:

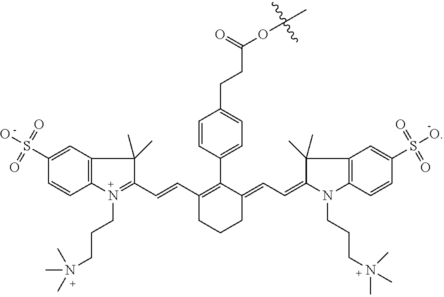

In some embodiments, B is selected from the group consisting of a biocompatible polypeptide and a biocompatible polyester, each of which is substituted by one or more C groups and one or more -D-E groups. In some embodiments, B is selected from the group consisting of polylysine, polylactic acid, poly(lactic-co-glycolic acid), polyaspartic acid, polyglutamic acid, and polyglutamic acid-poly(ethylene glycol) copolymer, each of which is substituted by one or more C groups and one or more -D-E groups. In some embodiments, B is polylysine substituted by one or more C groups and one or more -D-E groups. In some embodiments, the polylysine is ε-poly-L-lysine substituted by one or more C groups and one or more -D-E groups. In some embodiments, B is:

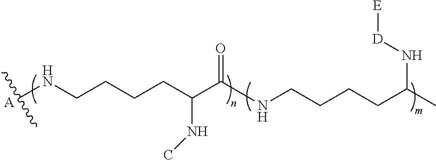

wherein
[symbol] indicates the bond between A and B; and n is an integer from 5 to 30.

In some embodiments, C is an anionic group of the following formula:

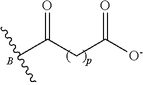

wherein:
[symbol] indicates the bond between C and B; and p is an integer from 1 to 10.

In some embodiments, D is a linking group of the following formula:

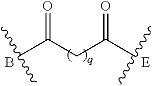

wherein: [symbol] indicates the bond between D and B or the bond between D and E; and q is an integer from 1 to 10. In some embodiments, q is an integer from 1 to 5.

In some embodiments, E is selected from the group consisting of an iron chelating group, a lead chelating group, a copper chelating group. an arsenic chelating group, a mercury chelating group, and a manganese chelating group. In some embodiments, E is an iron chelating group. In some embodiments, E is selected from the group consisting of dimercaptosuccinic acid, dimercaprol, ethylenediaminetetraacetic acid, p-aminosalicylic acid, D-penicillamine, deferoxamine, deferiprone, and deferasirox. In some embodiments, E is deferoxamine (DFO). In some embodiments, B is polylysine and E is an iron chelating group, wherein the polylysine is substituted by one or more C groups and one or more -D-E groups. In some embodiments, the polylysine is ε-poly-L-lysine (i.e., EPL) substituted by one or more C groups and one or more -D-E groups. In some embodiments, B is ε-poly-L-lysine and E is deferoxamine (i.e., DFO), wherein the ε-poly-L-lysine is substituted by one or more C groups and one or more -D-E groups.

In some embodiments, the molar ratio (i.e., stoichiometry) of the metal chelating agent E (e.g., an iron chelating agent) to the biocompatible polymer B in the compounds of Formula I is from about 10:1 to about 1:1, for example, about 10:1 to about 2:1, about 10:1 to about 4:1, about 10:1 to about 6:1, about 10:1 to about 8:1, about 8:1 to about 1:1, about 8:1 to about 2:1, about 8:1 to about 4:1, about 8:1 to about 6:1, about 6:1 to about 1:1, about 6:1 to about 2:1, about 6:1 to about 4:1, about 4:1 to about 1:1, about 4:1 to about 2:1, or about 2:1 to about 1:1. In some embodiments, the molar ratio (i.e., stoichiometry) of the metal chelating agent E (e.g., an iron chelating agent) to the biocompatible polymer B in the compounds of Formula I is about 2:1, about 4:1, about 6:1, or about 8:1.

In some embodiments, the hydrogel comprises a hyaluronic acid substrate. The term “hyaluronic acid substrate” encompasses all variants and combinations of variants of hyaluronic acid, or hyaluronan, of various chain lengths and charge states, as well as with various chemical modifications. In some embodiments, the hyaluronic acid substrate is a chemically unmodified hyaluronic acid or hyaluronate salt. In some embodiments, the hyaluronic acid substrate is sodium hyaluronate. In some embodiments, the hyaluronic acid substrate is hyaluronic acid (HA). HA is composed of D-glucuronic acid and D-N-acetylglucosamine, and is a disaccharide polymer in which β-1,4 and β-1,3 glucosidic bonds are alternately bonded.
In some embodiments, the hyaluronic acid substrate has an average molecular weight in the range of 0.1-10 MDa. In some embodiments, the hyaluronic acid substrate has an average molecular weight in the range of 0.8-5 MDa. In some embodiments, the hyaluronic acid substrate has an average molecular weight in the range of more preferably 1.5-3 MDa. In some embodiments, the hyaluronic acid substrate has an average molecular weight in the range of 2-3 MDa. In some embodiments, the hyaluronic acid substrate is obtained from non-animal origin. In some embodiments, the hyaluronic acid substrate is obtained from bacteria.
In some embodiments, the HA is crosslinked. In some embodiments, the hyaluronic acid is crosslinked with one or more polyfunctional cross-linking agents selected from the group consisting of divinyl sulfone, multiepoxides and diepoxides. In some embodiments, the polyfunctional cross-linking agents is selected from the group consisting of 1,4-butanediol diglycidyl ether (BDDE), 1,2-ethanediol diglycidyl ether (EDDE) and diepoxyoctane. In some embodiments, the polyfunctional cross-linking agent is 1,4-butanediol diglycidyl ether (BDDE). It is desirable that the one or more polyfunctional cross-linking agents provide ether cross-links.

In some embodiments, the hyaluronic acid product is single cross-linked. A single cross-linked product has the advantage of being chemically well defined. In some embodiments, the shaped hyaluronic acid product is multiple cross-linked. In specific embodiments, the shaped hyaluronic acid product is cross-linked with ether cross-links. Ether cross-linked hyaluronic acid gel products according to the disclosure are stable and can readily be sterilized, e.g. autoclaved.

In some embodiments, the hydrogel further comprises a poloxamer. A poloxamer is a nonionic triblock copolymer composed of a central hydrophobic chain of polyoxypropylene (poly(propylene oxide) (PPO)) flanked by two hydrophilic chains of polyoxyethylene (poly(ethylene oxide) (PEO)). In some embodiments, the poloxamer is selected from the group consisting of Pluronic L31, L35, F38, L42, L43, L44, L61, L62, L63, L64, P65, F68, L72, P75, F77, L81, P84, P85, F87, F88, L92, F98, L101, P103, P104, P105. F108, L121, L122, L123, F127, 10R5, 10R8, 12R3, 17R1, 17R2, 17R4, 17R8, 22R4, 25R1, 25R2, 25R4, 25R5, 25R8, 31R1, 31R2, 31R, and a mixture thereof. In some embodiments, the poloxamer is poloxamer 407 (Pluronic F127). Pluronic R F127 poloxamer is a synthetic triblock copolymer (PEO₉₉-PPO₆₇-PEO₉₉). Pluronic F127 and HA were selected as materials for the injectable hydrogel formulation because they are FDA-approved and are widely used in various fields including drug delivery systems. Pluronic F127 has a unique thermosensitive sol-gel transition facilitated by a micellar mechanism when concentrated in water. HA is used clinically as a dermal filler and drug delivery vehicle due to its superior biocompatibility, minimal toxicity, and low inflammatory properties. Thus, HA was implemented into the hydrogel to control the initial burst release of F127.

In some embodiments, the composition comprises from about 15 wt % to about 45 wt % of the iron chelator. In some embodiments, the composition comprises from about 25 wt % to about 35 wt % of the iron chelator. In some embodiments, the composition comprises from about 28 wt % to about 32 wt % of the iron chelator. In, the composition comprises about 15 wt %, about 16 wt %, about 17 wt %, about 18 wt %, about 19 wt %, about 20 wt %, about 21 wt %, about 22 wt %, about 23 wt %, about 24 wt %, 25 wt %, about 26 wt %, about 27 wt %, about 28 wt %, about 29 wt %, about 30 wt %, about 31 wt %, about 32 wt %, about 33 wt %, about 34 wt %, 35 wt %, about 36 wt %, about 37 wt %, about 38 wt %, about 39 wt %, about 40 wt %, about 41 wt %, about 42 wt %, about 43 wt %, about 44 wt %, or 45 wt % of the iron chelator. In some embodiments, the composition comprises from about 30 wt % of the iron chelator. In some embodiments, the composition comprises from about 30 wt % of DFO-NP.

In some embodiments, the composition comprises from about 1 wt % to about 20 wt % of the hyaluronic acid substrate. In some embodiments, the composition comprises from about 5 wt % to about 15 wt % of the hyaluronic acid substrate. In some embodiments, the composition comprises from about 6 wt % to about 10 wt % of the hyaluronic acid substrate. In, the composition comprises about 1 wt %, about 2 wt %, about 3 wt %, about 4 wt %, about 5 wt %, about 6 wt %, about 7 wt %, about 8 wt %, about 9 wt %, about 10 wt %, 11 wt %, about 12 wt %, about 13 wt %, about 14 wt %, about 15 wt %, about 16 wt %, about 17 wt %, about 18 wt %, about 19 wt %, or about 20 wt %, of the hyaluronic acid substrate. In some embodiments, the composition comprises from about 7 wt % of the hyaluronic acid substrate. In some embodiments, the composition comprises from about 7 wt % of HA.

In some embodiments, the composition comprises from about 15 wt % to about 45 wt % of the poloxamer. In some embodiments, the composition comprises from about 25 wt % to about 35 wt % of the poloxamer. In some embodiments, the composition comprises from about 28 wt % to about 32 wt % of the poloxamer. In, the composition comprises about 15 wt %, about 16 wt %, about 17 wt %, about 18 wt %, about 19 wt %, about 20 wt %, about 21 wt %, about 22 wt %, about 23 wt %, about 24 wt %, 25 wt %, about 26 wt %, about 27 wt %, about 28 wt %, about 29 wt %, about 30 wt %, about 31 wt %, about 32 wt %, about 33 wt %, about 34 wt %, 35 wt %, about 36 wt %, about 37 wt %, about 38 wt %, about 39 wt %, about 40 wt %, about 41 wt %, about 42 wt %, about 43 wt %, about 44 wt %, or 45 wt % of the poloxamer. In some embodiments, the composition comprises from about 30 wt % of the poloxamer. In some embodiments, the composition comprises from about 30 wt % of F127.

In some embodiments, the composition comprises from about 1 wt % to about 70 wt % of water. In some embodiments, the composition comprises from about 5 wt % to about 15 wt % of water. In some embodiments, the composition comprises from about 10 wt % to about 25 wt % of water. In some embodiments, the composition comprises from about 20 wt % to about 35 wt % of water. In some embodiments, the composition comprises from about 30 wt % to about 45 wt % of water. In some embodiments, the composition comprises from about 40 wt % to about 55 wt % of water. In some embodiments, the composition comprises from about 50 wt % to about 65 wt % of water. In, the composition comprises about 2 wt %, about 4 wt %, about 6 wt %, about 8 wt %, about 10 wt %, about 12 wt %, about 14 wt about 16 wt %, about 18 wt %, about 20 wt %, about 20 wt %, about 20 wt %, about 20 wt %, about 20 wt %, about 30 wt %, about 32 wt %, about 34 wt %, about 36 wt %, about 38 wt %, about 40 wt %, about 42 wt %, about 44 wt %, about 46 wt %, about 48 wt %, about 50 wt %, about 52 wt %, about 54 wt %, about 56 wt %, about 58 wt %, about 60 wt %, about 62 wt %, about 64 wt %, about 66 wt %, about 66 wt %, about 70 wt %, of water. In some embodiments, the composition comprises from about 33 wt % of water.

In some embodiments, the composition comprises 30 wt % poloxamer, 7 wt % HA, 30 wt % DFO-NP, and 33 wt % water.

### Methods of Treatment

In some embodiments, the disclosure provides a sustained release, injectable composition for iron chelation therapy in a human patient comprising an iron chelation agent and a hydrogel, said composition capable of release over a two week period. In some embodiments, the disclosure provides a sustained release, injectable composition for iron chelation therapy in a human patient comprising an iron chelation agent and a hydrogel, said composition is released over a two week period.

Some embodiments provide a method of treating iron overload in a patient in need thereof. The method includes administering an effective amount of a composition described herein to the patient. Other embodiments provide a method of treating acute kidney injury in a patient in need thereof. See for example, Leaf and Swinkels, Catalytic Iron and Acute Kidney Injury, Am J Physoi Renal Physiol 2016 Nov. 1; 311 (5): F871-F876. The method includes administering an effective amount of a composition described herein to the patient having acute kidney injury.

The present application further provides methods of chelating metal ions in a sample (e.g. a cell sample or a tissue sample) or a subject, comprising contacting the sample with, or administering to the subject, a compound provided herein or (e.g., a compound of Formula I) or a pharmaceutically acceptable salt thereof.

As used herein, the term “metal ions” refers to free metal ions or metal ions bound to low affinity ligands (e.g., citrate), or a combination thereof, in a sample (e.g., a cell sample or tissue sample) or a subject.

As used herein, the term “subject” or “patient” refers to any animal, including mammals. Example subjects include, but are not limited to, mice, rats, rabbits, dogs, cats, swine, cattle, sheep, horses, primates, and humans. In some embodiments, the subject is a human. In some embodiments, the method comprises administering to the subject a therapeutically effective amount of a composition described herein.

In some embodiments, the method is a method of chelating metal ions in a cell or tissue sample, comprising contacting the cell sample or tissue sample with a composition described herein. In some embodiments, the contacting forms a metal-compound chelate.

The present disclosure further provides a method of reducing the amount of free metal ions in a cell or tissue sample, comprising contacting the cell or tissue sample with a composition described herein. In some embodiments, the contacting forms a metal-compound chelate, thereby reducing the amount of free metal ions in the cell or tissue sample.

The present disclosure further provides a method of chelating metal ions in a subject, comprising administering to the subject a therapeutically effective amount of a composition described herein. In some embodiments, the metal ions are free metal ions or metal ions bound to low affinity ligands (e.g., citrate).

The present disclosure further provides a method of reducing the amount of free metal ions in a subject, comprising administering to the subject a composition described herein.

The present disclosure further provides a method of reducing the amount of metal ions (e.g., free metal ions or metal ions bound to low affinity ligands (e.g., citrate)), in the bloodstream of a subject in need thereof. In some embodiments, the method is a method of reducing the amount iron ions (e.g., free iron irons or iron ions bound to low affinity ligands (e.g., citrate)) in the bloodstream of a subject in need thereof. In some embodiments, the subject has been determined to have high levels of iron ions (e.g., free iron irons or iron ions bound to low affinity ligands (e.g., citrate)) in the bloodstream compared to a subject having normal levels of iron ions in the bloodstream.

The present disclosure further provides a method of reducing iron in a subject in need thereof, comprising administering to the subject a composition described herein. In some embodiments, the method is a method of reducing iron overload in a subject in need thereof.

The present disclosure further provides a method of reducing the amount of metal ions bound to low affinity ligands in a subject, comprising administering to the subject a composition described herein.

The present disclosure further provides a method of treating a disease associated with an abnormal amount of metal ions in a subject. In some embodiments, the disease is associated with an abnormal amount of free metal ions, an abnormal amount of metal ions bound to low affinity ligands (e.g., citrate), or a combination thereof. In some embodiments, the method comprises administering to the subject a compound provided herein, or a pharmaceutically acceptable salt thereof.

In some embodiments, the disease is associated with an abnormally high amount of metal ions in the subject (e.g., free metal ions, metal ions bound to low affinity ligands, or a combination thereof) compared to a subject having normal levels of metal ions. In some embodiments, the disease is associated with an abnormally high amount of free metal ions in the subject, compared to a subject having normal levels of free metal ions. In some embodiments, the disease is associated with an abnormally high amount of metal ions bound to low affinity ligands in the subject, compared to a subject having normal levels of metal ions bound to low affinity ligands in the subject. In some embodiments, the disease is associated with an abnormally high amount of a combination of free metal ions and metal ions bound to low affinity ligands in the subject, compared to a subject having normal levels of free metal ions and metal ions bound to low affinity ligands.

In some embodiments, the abnormal amount of metal ions in the subject refers to about 5% to about 100% increased concentration of metal ions in the subject compared to the concentration of metal ions in a normal subject, for example, about 5% to about 100%, about 5% to about 75%, about 5% to about 50%, about 5% to about 25%, about 5% to about 10%, about 10% to about 100%, about 10% to about 75%, about 10% to about 50%, about 10% to about 25%, about 25% to about 100%, about 25% to about 75%, about 25% to about 50%, about 50% to about 100%, about 50% to about 75%, or about 75% to about 100%, increased concentration of metal ions in the subject compared to the concentration of metal ions in a normal subject.

In some embodiments, the abnormal amount of metal ions in the subject refers to about 2 fold to about 10 fold increased concentration of metal ions in the subject compared to the concentration of metal ions in a normal subject, for example, about 2 fold to about 10 fold, about 2 fold to about 8 fold, about 2 fold to about 5 fold, about 2 fold to about 3 fold, about 3 fold to about 10 fold, about 3 fold to about 8 fold, about 3 fold to about 5 fold, about 5 fold to about 10 fold, about 5 fold to about 8 fold, or about 8 fold to about 10 fold, increased concentration of metal ions in the subject compared to the concentration of metal ions in a normal subject.

Methods of determining the concentration of metal ions in a subject are routine in the art and include, for example, measuring metal ions in a cell sample (e.g., NIR microscopy) or tissue sample (e.g., a biopsy sample by NIR spectroscopy) and/or measuring metal ions in the subject using an imaging technique (e.g., magnetic resonance imaging and/or optical fluorescence imaging).

The present application further provides a method of treating a disease associated with an abnormal amount of free metal ions in a subject. In some embodiments, the method comprises administering to the subject a compound provided herein, or a pharmaceutically acceptable salt thereof.

In some embodiments, the disease is associated with an abnormal amount of iron ions, an abnormal amount of lead ions, an abnormal amount of copper ions, an abnormal amount of arsenic ions, an abnormal amount of manganese ions, an abnormal amount of cadmium ions, an abnormal amount of nickel ions, an abnormal amount of chromium ions, an abnormal amount of gold ions, or an abnormal amount of antimony ions in the subject, or any combination thereof. In some embodiments, the disease is associated with an abnormal amount of iron ions, an abnormal amount of lead ions, or an abnormal amount of copper ions in the subject, or any combination thereof.

In some embodiments, the disease is associated with an abnormal amount of iron ions, an abnormal amount of lead ions, or an abnormal amount of copper ions in the subject. In some embodiments, the disease is associated with an abnormal amount of iron ions in the subject.

In some embodiments, the disease is selected from the group consisting of transfusion hemosiderosis (e.g., resulting from blood transfusions in a subject having one or more diseases selected from the group consisting of thalassemia, myelodysplastic syndrome, sickle cell anemia, and Blackfan Diamond anemia), hemochromatosis (e.g., hereditary or acquired), Wilson's disease, copper poisoning, and heavy metal poisoning (e.g., lead poisoning, mercury poisoning, cadmium poisoning, arsenic poisoning, manganese poisoning, and the like).

As used herein, the term “treating” or “treatment” refers to one or more of (1) inhibiting the disease: for example, inhibiting a disease, condition or disorder in an individual who is experiencing or displaying the pathology or symptomatology of the disease, condition or disorder (i.e., arresting further development of the pathology and/or symptomatology); and (2) ameliorating the disease: for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

The term “effective amount” as used herein refers to the amount of an agent(s) needed to alleviate at least one or more symptom of the disease or disorder and relates to a sufficient amount of pharmacological composition to provide the desired effect, for example, relieving iron overload. An effective amount as used herein would also include an amount sufficient to delay the development of a symptom of the disease, alter the course of a symptom of disease (for example but not limited to, slow the progression of a symptom of the disease), or reverse a symptom of disease. Thus, it is not possible to specify the exact “effective amount.”

The compositions described herein are isolated agents, meaning that the agents are substantially pure and are essentially free of other substances with which they may be found in nature or in vivo systems to an extent practical and appropriate for their intended use. In particular, the agents are sufficiently pure and are sufficiently free from other constituents so as to be useful in, for example, producing pharmaceutical preparations. Because an isolated composition may be admixed with a pharmaceutically acceptable carrier in a pharmaceutical preparation, the compositions, i.e., the active agents, may comprise only a small percentage by weight of the preparation.

In some embodiments, the compositions described herein are administered to a subject preferably by injection administration or infusion instillation. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions for use in the methods described herein are administered by intravenous infusion or injection. The phrases “parenteral administration” and “administered parenterally” as used herein, refer to modes of administration other than enteral and topical administration, usually by injection. The phrases “systemic administration,” “administered systemically,” “peripheral administration,” and “administered peripherally” as used herein refer to the administration of an agent other than directly into a target site, tissue, or organ, such that it enters the subject's circulatory system and, thus, is subject to systemic metabolism and other like processes.

For the clinical use of the methods described herein, administration of an agent or composition can include formulation into pharmaceutical compositions or pharmaceutical formulations for parenteral administration, e.g., intravenous or other mode of administration. In some embodiments, an agent can be administered along with any pharmaceutically acceptable carrier compound, material, or composition which results in an effective treatment in the subject. Thus, a pharmaceutical formulation for use in the methods described herein can contain an agent as described herein in combination with one or more pharmaceutically acceptable ingredients.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or diluent, excipient, solvent, involved in maintaining the stability, solubility, or activity of the composition. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (19) pH buffered solutions; (20) polyesters, polycarbonates and/or polyanhydrides; (21) bulking agents, such as polypeptides and amino acids (22) serum components, such as serum albumin, HDL and LDL; (23) C2-C 12 alcohols, such as ethanol; and (24) other non-toxic compatible substances employed in pharmaceutical formulations. Release agents, coating agents, preservatives, and antioxidants can also be present in the formulation. The terms such as “excipient,” “carrier,” “pharmaceutically acceptable carrier,” or the like are used interchangeably herein. The agents described herein can be specially formulated for administration of the compound to a subject in liquid form, including those adapted for parenteral administration, for example, by subcutaneous, intramuscular, intravenous or epidural injection as, for example, a sterile solution or suspension, or sustained-release formulation. Parenteral dosage forms of an agent can also be administered to a subject by various routes, including, but not limited to, subcutaneous, intravenous (including bolus injection), intramuscular, and intraarterial. Since administration of parenteral dosage forms typically bypasses the patient's natural defences against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions. Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Processes for Preparing the Compositions

In yet another aspect, the disclosure includes a method of making a sustained-release, injectable composition useful iron chelation therapy.

“Sustained release” or “extended release” means that the iron chelation therapy or the iron chelation agent is released from the composition at a controlled rate so that therapeutically beneficial blood levels (but below toxic levels) of the iron chelation agent is maintained over an extended period of time. Alternatively, “sustained release” or “extended release” means that the desired pharmacologic effect is maintained over an extended period of time. Experiments using the compositions described herein have surprisingly found that the duration delivery of iron chelation agent is longer than expected. In the experiments described herein, however, it was surprisingly discovered that the release of the iron chelation agent from the experimental injected compositions was for over a period of 1 to 30 days, at least 2 weeks, and in certain experiments longer than 2 weeks. In some cases, the iron chelation effect was at least about 2 weeks, thus providing the possibility of fewer dosing administrations, which was heretofore not thought to be possible.

In some embodiments, the sustained release of the iron chelation agent from the compositions disclosed herein is over a period of 1 day to about 30 days. In some embodiments, the sustained release of the iron chelation agent from the compositions disclosed herein is from about 10 days to about 21 days. In some embodiments, the sustained release of the iron chelation agent from the compositions disclosed herein is from about 14 days to about 21 days. In some embodiments, the sustained release of the iron chelation agent from the compositions disclosed herein is from about 10 days to about 15 days. In some embodiments, the sustained release of the iron chelation agent from the compositions disclosed herein is at least 5 days. In some embodiments, the sustained release of the iron chelation agent from the compositions disclosed herein is at least 10 days. In some embodiments, the sustained release of the iron chelation agent from the compositions disclosed herein is at least 12 days. In some embodiments, the sustained release of the iron chelation agent from the compositions disclosed herein is at least 14 days (2 weeks). In some embodiments, the sustained release of the iron chelation agent from the compositions disclosed herein is at least 16 days. In some embodiments, the sustained release of the iron chelation agent from the compositions disclosed herein is at least 18 days. In some embodiments, the sustained release of the iron chelation agent from the compositions disclosed herein is at least 20 days. In some embodiments, the sustained release of the iron chelation agent from the compositions disclosed herein is at least 21 days (3 weeks). In some embodiments, the sustained release of the iron chelation agent from the compositions disclosed herein is at least 22 days. In some embodiments, the sustained release of the iron chelation agent from the compositions disclosed herein is at least 24 days. In some embodiments, the sustained release of the iron chelation agent from the compositions disclosed herein is at least 26 days. In some embodiments, the sustained release of the iron chelation agent from the compositions disclosed herein is at least 28 days. In some embodiments, the sustained release of the iron chelation agent from the compositions disclosed herein is at least 30 days.

In some embodiments, the disclosure includes a method of preparing a sustained-release, injectable composition useful for metal chelation therapy comprising the steps:
a) cross-linking a hyaluronic acid substrate;
b) making the cross-linked hyaluronic acid substrate into particles;
c) adding the particles of cross-linked a hyaluronic acid substrate into a solution of metal chelator; and
d) further adding a poloxamer to the resulting solution of step c).

In some embodiments, the disclosure includes a method of preparing a metal chelating hydrogel composition comprising the steps:
a) cross-linking a hyaluronic acid substrate;
b) making the cross-linked hyaluronic acid substrate into particles;
c) lyophilizing the particles;
d) adding the particles of cross-linked hyaluronic acid substrate into a solution of metal chelator; and
e) further adding a poloxamer to the resulting solution of step d);
f) isolating the hydrogel comprising the metal chelator loaded into cross-linked hyaluronic acid substrate particles integrated with a poloxamer.

In some embodiments, the hyaluronic acid substrate is HA. In some embodiments, the metal chelator is DFO or DFO-NP. In some embodiments, the poloxamer is F127.

In some embodiments, the injectable hydrogel was prepared by integrating crosslinked-hyaluronic acid (xHA) into Pluronic F127 (F127), both FDA-approved, to load a high dose of DFO-NP and to diminish the initial burst release. The hydrogel showed thermosensitive rheological properties, and its release kinetics were evaluated longitudinally by using a NIR fluorescence imaging system. This sustained release hydrogel formulation improves therapeutic efficacy while minimizing toxicity by offering a long-term release of iron nanochelators with a short-term residence in non-target tissues, which is beyond the capability of current small molecule chelators. Considering the need for lifelong administrations of chelators in patients with iron overload, hydrogel-based nanochelators offer significant advantages over the current chelation therapies. This injectable hydrogel formulation showed a thermosensitive sol-gel transition at body temperature and provided a prolonged release of renal clearable iron nanochelators over 2 weeks, resulting in a half-life 47-fold longer than that of the nanochelator alone.

### EXAMPLES

Materials: Epsilon-poly-L-lysine (EPL: MW ˜3,900) was purchased from BOC Sciences (Shirly, NY). Ninhydrin agent, hyaluronic acid (100 kDa), and succinic anhydride (SA) were purchased from Acros Organics (Morris Plains, NJ). Ethyl acetate (EA), deuterium oxide (D₂O), anhydrous dimethyl sulfoxide (DMSO), pluronic F127, 4-(4,6-dimethoxy-1,3,5-triazin-2-yl)-4-methyl-morpholinium chloride (DMTMM), ferric chloride, sodium acetate, and sodium hydroxide were purchased from Sigma-Aldrich (Saint Louis, MO). DFO and assay reagent kits (for aspartate aminotransferase, alanine aminotransferase, and serum creatinine) were purchased from Cayman Chemical (Ann Arbor, MI).

### Example 1: Synthesis and Characterization of DFO-NP

DFO-NP was prepared as described in WO2018/147894 (herein incorporated by reference in its entirety).

Prior to designing the injectable hydrogel formulation, the DFO-NP was prepared. Zwitterionic NIR fluorophore (ZW800-1C) was conjugated to ε-poly-L-lysine (ZW-EPL⁺) for monitoring of the in vivo behaviour of DFO-NP. All of the primary amines of ZW-EPL⁺ were succinylated (ZW-EPL⁻) to convert the side chains into negatively charged carboxylate groups and hence enable DFO conjugation via amide bonding on ZW-EPL⁻ (DFO-NP). See Scheme 1 below.

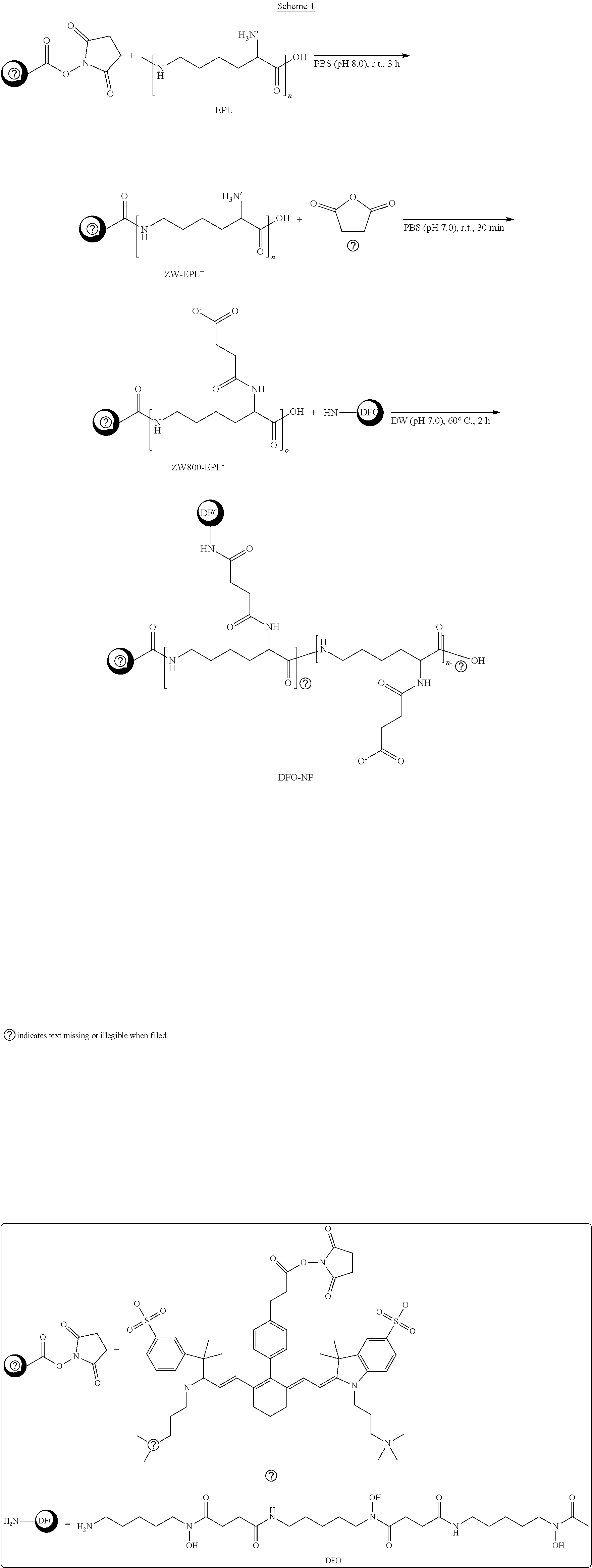

### Synthesis of ZW800-1C Conjugated EPL (ZW-EPL⁺):

To prepare ZW800-1C conjugated EPL. 1 g of epsilon poly-L-lysine (EPL) was dissolved in 100 mL of phosphate buffer saline. The pH of the EPL solution was adjusted to around 8.0 with 6 M NaOH solution. ZW800-1C-succinic amide ester (ZW800-1C-NHS) was dissolved in DMSO at a concentration of 25 mg mL⁻¹. 5 mL of ZW800-1C-NHS solution was dropped into the EPL solution with vigorous stirring at room temperature. After 3 hours, the reaction mixture was added into 1 L of acetone/ethyl acetate (EA) (4/1) to precipitate ZW800-EPL. The mixture was centrifuged at 3000 rpm for 15 min at 4° C. The supernatant was discarded, and the precipitant was redissolved in DW (>50 mL) and re-precipitated in 1 L of acetone/EA. The precipitation was repeated two more times to complete the purification. The final precipitant was dried in a vacuum overnight.

### Synthesis of Succinylated ZW800-EPL (ZW-EPL⁻):

ZW800-EPL⁺ (1 g, 0.2 mmol) and EPL (19 g, 4.75 mmol) were dissolved together in 2 L of PBS. 45 g of succinic acid (450 mmol) in 180 mL of DMSO (250 mg/mL) was added to the ZW-EPL solution. The pH of the reaction mixture was adjusted to around 7.0 with 6 M NaOH solution as needed. The reaction mixture was stirred for 30 min at room temperature. After, the succinylation ratio was confirmed by the ninhydrin test. For purification, the reaction mixture was precipitated with the same procedure described in the previous section.

### Synthesis of Renal Clearable Nanochelator (DFO-NP):

50 g of deferoxamine (76 mmol) was dissolved in 500 mL of DW. The DFO solution was neutralized by adding of 6 M NaOH solution. 20 g of ZW-EPL⁻ (2.5 mmol) was dissolved in 1.25 L of DW. 42 g of DMTMM and the prepared DFO solution were added to the ZW-EPL⁻ solution. The reaction mixture was stirred for 2 h at 60° C., followed by dialysis performed against DW with a cellulose dialysis membrane of MWCO 6-8 kDa. After dialysis, the solution was lyophilized.

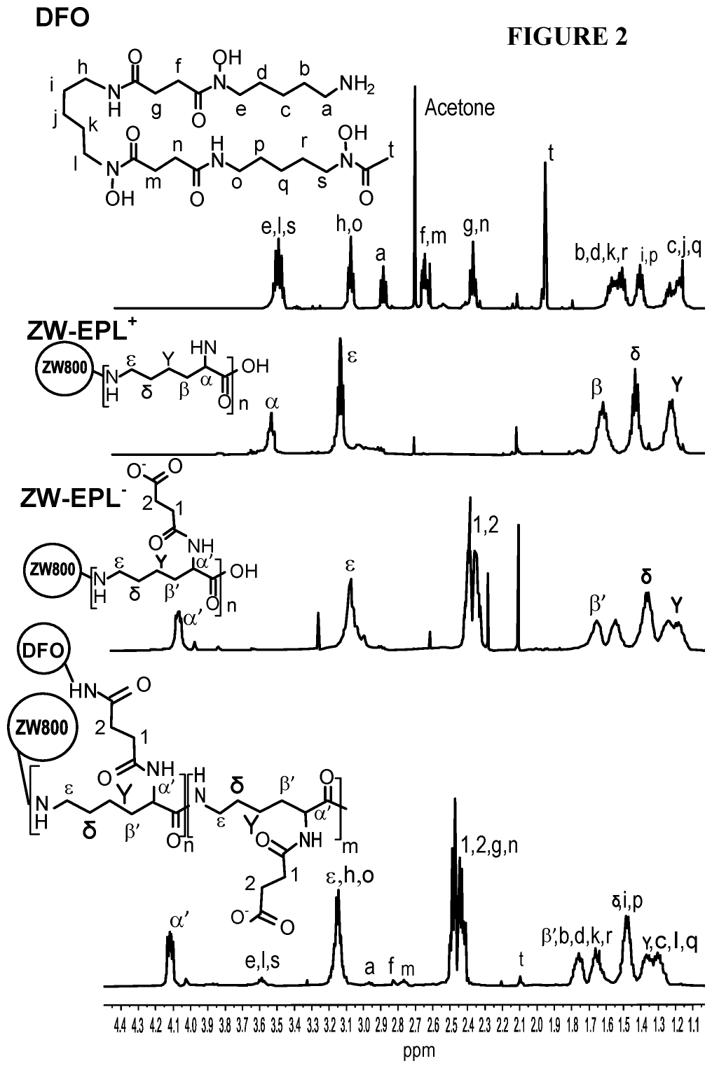

¹H-NMR analysis: To determine the DFO conjugation ratio on the nanochelator, 10 mg of DFO-NP was dissolved in 600 μL of D₂O. ¹H-NMR spectroscopy was performed with a Varian 500 MHz spectrometer. The number of DFOs on succinylated EPL was calculated by comparing the peak integration values of succinylated EPL's protons at 4.1 ppm and DFO's position e,l,s protons at 3.6 ppm (FIG. 2).

Each product was analyzed and the number of DFOs on DFO-NP was quantified to be ˜1.0 by nuclear magnetic resonance (NMR) spectroscopy. See FIG. 2. The peaks at 4.12 ppm (position α′) and 3.6 ppm (position e,l,s) correspond to the protons of EPL and DFO, respectively.

Optical properties of DFO-NP: DFO-NP was dissolved in DW at a concentration of 100 μM. Absorbance and fluorescence emission spectra were observed from 500 nm to 1000 nm with a UV/Vis/NIR spectrometer (USB2000, Ocean Insight, Dunedin, FL). For the fluorescence emission spectrum, a 760 nm laser (Nawoo, Gwangju, Korea) was used for excitation.

Size-exclusion chromatography analysis: To measure the purity and hydrodynamic diameter (HD) of DFO-NP, size-exclusion chromatography (SEC) was performed with the Waters HPLC system consisting of a Waters e2695 separations module and Waters 2998 PDA detector. The column used was an Xbridge BEH 125 Å 3.5 μm (7.8×150 mm, Waters) SEC column. The mobile phase was isocratic with 10 mM PBS for 15 min at a flow rate of 0.75 mL min-1. Each component in the reaction mixture could be identified by its retention time and absorbance wavelength.

The standard calibration curve of HD was calculated by injecting 10 μl of protein standards containing aprotinin (6.5 kDa, 1.96 nm), ribonuclease (13.7 kDa, 3.28 nm), ovalbumin (44 kDa, 6.10 nm), and thyroglobulin (669 kDa, 9.6 nm) to the HPLC with the same mobile phase and flow rate described above. The partition coefficient, Kₐᵥ was obtained from the following equation (1):

$${K}_{av} ( {V}_{e} - {V}_{0} ) / ( {V}_{c} - {V}_{0} ) \qquad ( 1 )$$

where V₀, V_{c}, and Vₑ are column void volume, geometric column volume, and eluent volume, respectively. The HD of DFO-NP was calculated by the following equation (2):

$$HD = 3.778 {( \frac{0.4608 - {K}_{av}}{{K}_{av} + 0.008565} )}^{\frac{1}{2.65}} \qquad ( 2 )$$

The hydrodynamic diameter (HD) was analyzed by high-performance liquid chromatography (HPLC) with a size exclusive chromatography column. The HD of a NP is one of the key factors in predicting its clearance route, and HDs smaller than the renal threshold (6-8 nm) are preferred for renal clearance.^{[14]}

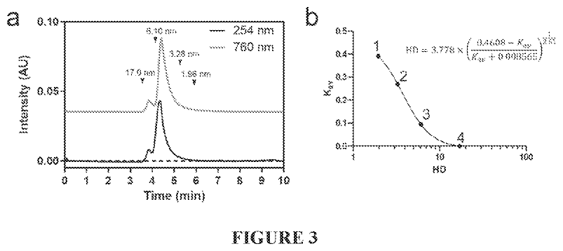

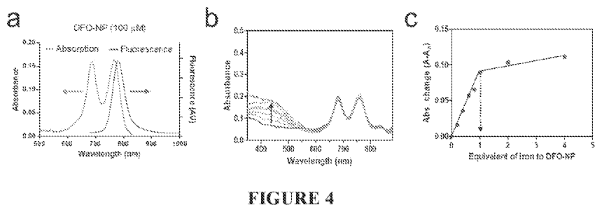

The HD of DFO-NP was calculated to be 5.2 nm when compared to the calibration curve of a known standard protein set, which suggested DFO-NP can be renal clearable. See FIG. 3. In addition, the purity of DFO-NP was confirmed to be 96% which suggests the absence of impurities such as unconjugated DFO and ZW800-1C. The optical properties and iron-binding efficacy of DFO-NP were evaluated by spectrophotometry.

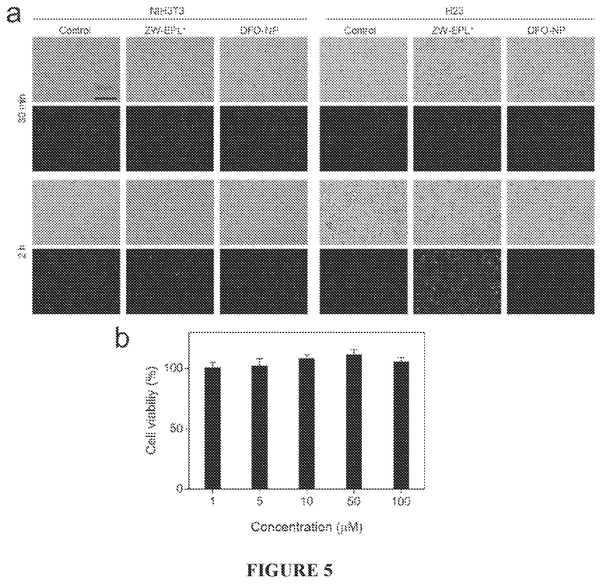

The maximum wavelengths of absorption and fluorescence emission for DFO-NP were 760 and 780 nm, respectively. See FIG. 4 (a) which allow the DFO-NP to be detected in a NIR channel with minimal tissue scattering and autofluorescence. The iron-binding efficacy of DFO-NP was confirmed in an in vitro iron-binding assay using ferric chloride (FeCl₃). Since the complex of DFO and Fe³⁺ ion absorbs wavelengths of 430 nm, the chelation of DFO-NP with iron could easily be observed by measuring UV-Vis absorbance.

The absorption value at 430 nm increased as the 5 μL FeCl₃ solutions (4 mM) were sequentially added to the DFO-NP solution (1000 μL, 100 UM). See FIG. 4 (b). After adding 25 μL of FeCl₃ solution, which is equimolar to the DFO-NP solution, it was shown that the increase of absorption at 430 nm was reduced (Figure S4c, Supporting Information). These results indicate that the stoichiometry value of DFO-NP is ˜1.0 due to DFO forming a monodentate complex with Fe³⁺ ions, which is consistent with the ¹H-NMR result for proton quantification.

### Example 2: In Vitro Iron-Chelating Effect of DFO-NP

To confirm the iron-chelating effect of DFO-NP, a solution of DFO-NP was prepared at a concentration of 100 μM. 5 μL of ferric chloride solution (4 mM) was added to the DFO-NP solution with continuous measurement of the absorbance change at 430 nm which is the absorption value of the Fe³⁺ and DFO complex. The functional stoichiometry was calculated based on the titration curve prepared by using the absorbance change at 430 nm.

Cell culture: NIH3T3 and H23 cells were cultured in Dulbecco's modified eagle medium (DMEM, Mediatech, Herndon, VA) that contained 10% fetal bovine serum and 1% penicillin-streptomycin. NIH3T3 and H23 were incubated in a 75 cm² tissue culture flask (Corning, NY) under 5% CO₂ at 37° C., respectively.

In vitro cellular uptake and cytotoxicity of DFO-NP: NIH3T3 (1×10⁴) and H23 (1×10⁴) cells were seeded in a 96-well plate (Corning, NY) and cultured for 24 h, respectively. Cells were washed with 200 μL of PBS two times. 100 μL of fresh complete medium containing ZW800-EPL+ (5 μM) or DFO-NP (5 μM) was added to each well. Cells were incubated for 30 min and 2 h. The cells without any treatments were used as a control. At predetermined time points, cells were washed with PBS, followed by being fixed with 4% paraformaldehyde. The fixed cells were observed using Cytation5 (BioTek, Winooski, VT) and NanoenTek JuLi Stage (Seoul, S. Korea) in a bright and NIR fluorescence channel.

For the cytotoxicity test, NIH3T3 cells were treated with DFO-NP ranging from 1 to 100 μM. After 24 h, NIH3T3 cells were washed with PBS twice. 100 μL of fresh complete medium was added to each well. Then, 10 μL of CCK8 solution was added to each well. After 4 h, the absorbance was measured at 450 nm using a microplate reader (Cytation5). All experiments were carried out with three replicates.

Next, an in vitro cellular uptake experiment was performed to confirm the nonsticky property of DFO-NP in cells. See FIG. 5 a. NIH3T3 and NCL-H23 (H23) cells were selected as representative normal and cancerous cells, respectively, and positively charged ZW-EPL⁺ was used as a positive control. In fluorescence microscope images, ZW-EPL⁺ treated cells showed strong NIR fluorescence signals after 2 h, while DFO-NP treated cells exhibited no signal, suggesting that the negatively charged surface of DFO-NP can minimize nonspecific cell uptake. In addition, the cytotoxicity of DFO-NP was tested in NIH3T3 cells by incubating with various concentrations of DFO-NP (1, 5, 10, 50, and 100 UM) for 24 h, showing there is no cytotoxicity associated with DFO-NP See FIG. 5 b.

### Example 3: Preparation and Characterization of the Injectable Hydrogels

To start the hydrogel formulation process, HA was crosslinked with 1,4-butanediol diglycidyl ether (BDDE). See Scheme 2 below.

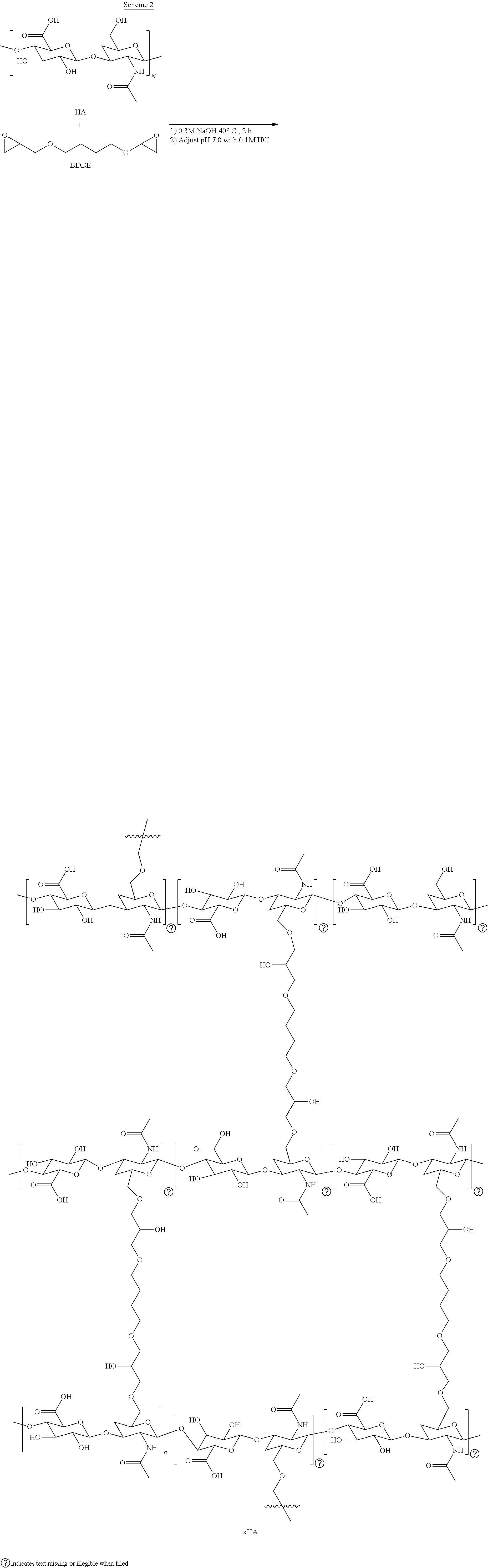

Preparation of crosslinked HA: 100 mg of HA was dissolved in 600 μL of 0.3 M NaOH solution. 20 μL of 1,4-Butanediol diglycidyl ether (BDDE) was added to the HA solution. The reaction mixture was vortexed for 1 min and incubated for 2 h at 40° C. Then the mixture was neutralized with 0.1 M HCl to a pH of 7.0. The neutralized reaction mixture was dialyzed against deionized water (DW) using a 6-8 kDa molecular weight cutoff (MWCO) cellulose dialysis membrane to remove residual BDDE. After dialysis, the HA hydrogel was broken down to an injectable particle size using a syringe with 18-23 G needles. The injectable cross-linked HA hydrogel particles (xHA) were then lyophilized.

Since crude xHA is barely injectable, it was crushed through 23-25 gauge needles by applying pressure, resulting in the crushed xHA particulates (HAPs) which could be injected via the syringe with a 23G needle. The HAPs were lyophilized and subsequently immersed into DFO-NP solution to load the DFO-NPs into HAPs. Dried DFO-NP loaded HAPs were added into Pluronic F127 solution for the final hydrogel formulation. The final weight percentages of DFO-NP, HAP, and Pluronic F127 are 30, 7, and 30 (denoted as DFO-NP/HA/F127; 30/7/30%), and the final concentration of DFO-NP in the formulation was 40 mM. To compare the effects of hybrid HA and F127 hydrogel, DFO-NP/HA; 30/7% and DFO-NP/F127; 30/30% were prepared as controls. All prepared formulations were injectable using a syringe with a 23G needle.

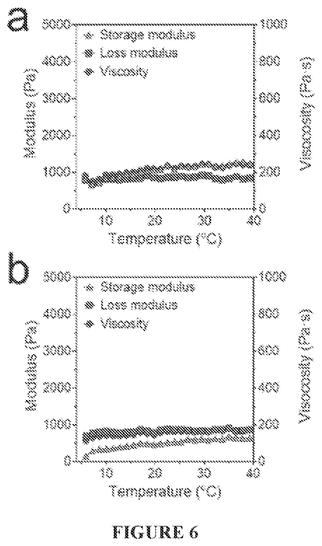

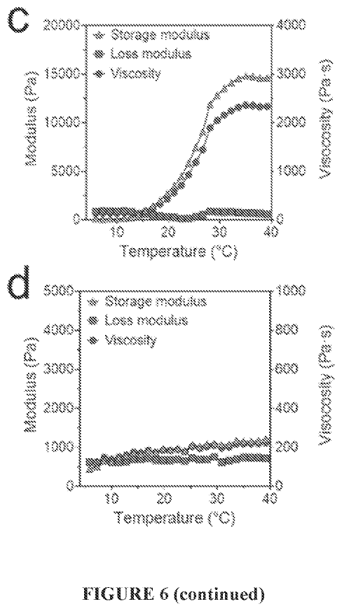

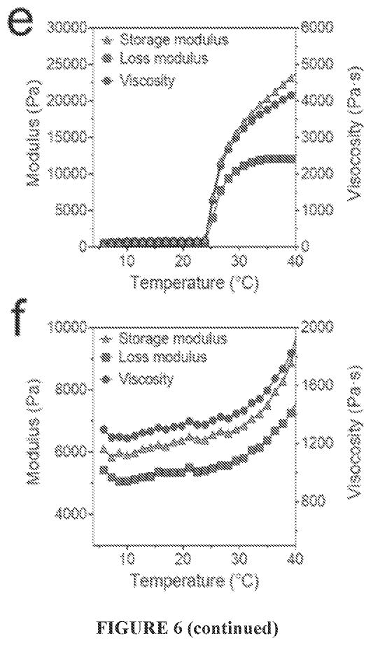

To evaluate the thermosensitivity of prepared hydrogels, rheological properties such as loss modulus, storage modulus, and viscosity were measured over a temperature ramp from 4 to 40° C. (FIG. 6). Since high content of drugs in thermosensitive hydrogels generally suppresses their thermo-gelling property, each corresponding hydrogel without DFO-NPs (HA; 7%, F127; 30%, and HA/F127; 7/30%) were also tested to confirm the effect of DFO-NPs on gelation. HA and DFO-NP/HA showed no significant change in rheological properties as the temperature ramped up (FIG. 6a,b), and the storage and loss moduli stayed below 1,000 Pa. The F127 showed a continuous increase of storage modulus and viscosity in a temperature range from 14 to 32° C. which indicates a thermosensitive sol-gel transition at 15° C. (FIG. 2c,d). In contrast, no thermo-responsive behavior in the DFO-NP/F127 was observed indicating that the high content of DFO-NP interrupted the hydrophobic aggregation of Pluronic micelles. Interestingly, both HA/F127 and DFO-NP/HA/F127 exhibited thermo-gelling properties (FIG. 6e,f). In HA/F127, all rheological parameters significantly increased above 25° C. which is likely attributed to a reinforcement by the HAP network. Despite the high content of DFO-NP, the moduli and viscosity values for DFO-NP/HA/F127 were higher than that of HA/F127 in the low-temperature range (<25° C.). In addition, DFO-NP/HA/F127 showed thermosensitive rheological changes above 30° C. and the storage modulus increased up to 8,500 Pa. These results suggest that DFO-NP/HA/F127 is not only injectable but also can form a firm hydrogel at body temperature due to high moduli.

### Example 4: In Vivo Release and Pharmacokinetics of DFO-NP Loaded Injectable Hydrogels

Preparation of DFO-NP loaded injectable hydrogels: DFO-NP, prepared as described in WO2018/147894 (herein incorporated by reference for the substance of its disclosure) was dissolved in DW at a concentration of 1 g mL⁻¹. 70 mg of xHA was added to 300 μL of DFO-NP solution to prepare DFO-NP loaded xHA. DFO-NP loaded xHA was lyophilized. The lyophilized DFO-NP loaded xHA was added to 1 mL of DW (DFO-NP/HA) or 1 mL of 30 wt % pluronic F127 solution (DFO-NP/HA/F127) on ice before using. 300 mg of DFO-NP was added to 1 mL of 30 wt % F127 (DFO-NP/F127).

Rheological characterization of hydrogels: The rheological properties of hydrogel formulations were analyzed using Haake Viscotester IQ Rheometer (Thermo Scientific, Germany) with a Peltier temperature-controlled bottom plate and a 25.0 mm stainless steel parallel plate measuring system All measurements were performed at 4-40° C. with a 0.5 mm gap. The temperature was changed by 0.03° C. s⁻¹. γ Strain and oscillating frequency were set at 0.01 and 1 Hz, respectively.

In vivo pharmacokinetics of DFO-NPs released from hydrogel formulations: Animals were housed in an AAALAC-certified facility and were studied under the supervision of MGH IACUC in accordance with the approved institutional protocol (2016N000136). Before injection of DFO-NP and DFO-NP loaded hydrogel formulations, six-week-old CD-1 mice (male: 25-30 g) from Charles River Laboratories (Wilmington, MA) were anesthetized with isoflurane & oxygen, and blood was sampled in capillary tubes (Fisher Scientific, Pittsburgh, PA) at time point 0 min by slightly cutting the end of the tail. For subcutaneous injection, DFO-NP was dissolved in DW at a concentration of 30 wt %, and DFO-NP/F127, DFO-NP/HA, and DFO-NP/HA/F127 were prepared as described above. The mice were separated into 4 groups (n=3). 100 μL of each formulation was injected subcutaneously into the back of mice. After injection, in vivo fluorescence images were taken using our NIR imaging system (K-FLARE) with an 800 nm channel at predetermined time points and blood samples were collected using capillary tubes at the same time. The fluorescence intensities of serum samples in capillary tubes were measured with Cytation5. 14 days after injection, mice were sacrificed to observe the biodistribution of DFO-NP and the organs (liver, lung, spleen, kidney, intestine, and bladder).

Iron chelation efficacy of DFO-NP HA F127. CD-1 mice (male: 30-35 g; Charles River Laboratories) were fed a high-iron diet (10,000 ppm Fe per kg) for 1 week. After, all mice were treated with facility chow (300 ppm Fe per kg) and administered saline, blank NP/HA/F127 (84 μmol/kg as NP), or DFO-NP/HA/F127 (84 μmol/kg as NP) by SC injection. At 3 weeks post administration, mice were euthanized, followed by heart, liver, and spleen collection for the analysis of iron contents by a non-heme iron colorimetric analysis using bathophenanthroline disulfonic acid.

Histological analysis of organs: Resected organs (heart, lung, liver, spleen, and kidney) were stored at −80° C. The frozen organs were fixed in 10% neutral buffered formalin and were dehydrated in ethanol, embedded in paraffin, and sectioned into slices (5 μm). Then, those sections were stained with hematoxylin and eosin (H&E) for pathology observation under the optical microscopic system (Cytation 5, BioTek Instruments).

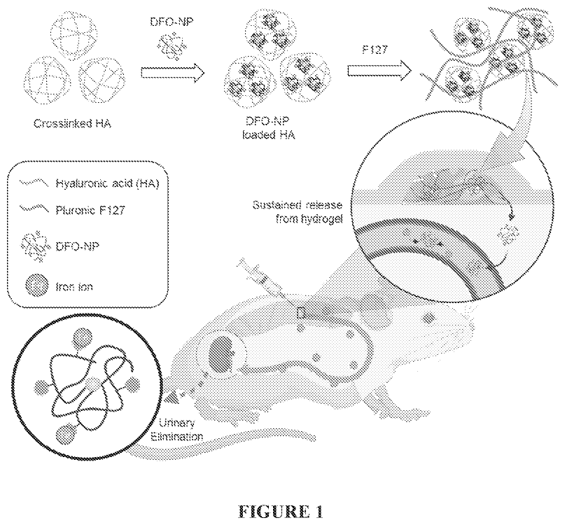

To evaluate the sustained-release property of the above hydrogel formulations, DFO-NP loaded injectable formulations and DFO-NP solution were subcutaneously injected into the back of mice (FIG. 1).

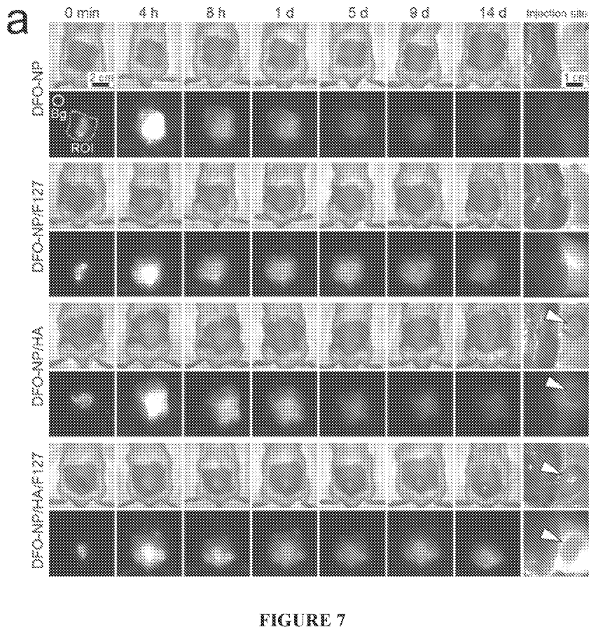

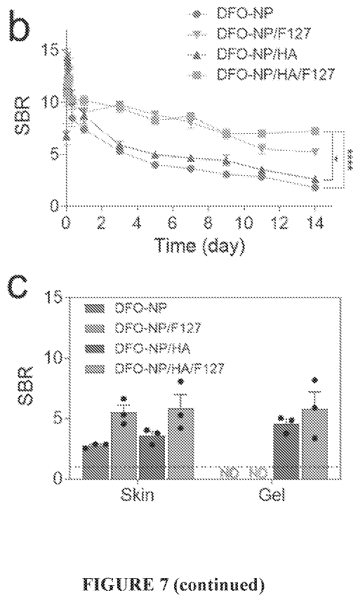

The injected dose of DFO-NP was 125 μmol kg-1 in all mice. The fluorescent signal of DFO-NP in mice was observed with a real-time NIR fluorescence imaging system (K-FLARE) for 14 days after injection (FIG. 7). The fluorescence signal in all experimental groups decreased rapidly 1 day after injection due to the initial burst release. In the DFO-NP solution injected mice, the fluorescence signal became negligible after 5 days and almost no fluorescent signal was observed after 14 days even after peeling off the skin at the injection site. The fluorescence intensity and signal-to-background ratio (SBR) of DFO-NP/F127 decreased slowly from 10 to 5 over 1 to 14 days, and there was no remaining hydrogel at the injection site. For DFO-NP/HA, the observed fluorescence signal as well as SBR were lower than those of DFO-NP/F127 after 1 day and decreased gradually over time. However, hydrogels were found at the injection sites after 14 days (white arrowheads in FIG. 3a). The fluorescence signal in the DFO-NP/HA/F127 group diminished more slowly than those of the DFO-NP and DFO-NP/HA groups, of which the SBR pattern was similar to that of the DFO-NP/F127 group (FIG. 7b). The SBR of DFO-NP/HA/F127 was maintained at around 7.0 from 9 days to 14 days, which was significantly higher than the other groups. In addition, the hydrogel still remained in the DFO-NP/HA/F127 group, and a relatively higher fluorescence intensity was observed around the hydrogel at the injection site after 14 days (FIG. 7a,b). For a more detailed comparison, we calculated the SBR of the skin and hydrogels at the injection sites after 14 days (FIG. 7c).

Although the initial burst release of DFO-NP could not be fully suppressed in the hydrogel formulations, it is worth noting that the rapid removal of non-transferrin bound iron, the predominant form of iron circulating in the blood, is important to prevent recurrence in patients being treated for iron overload. Therefore, indeed, the initial release of some portion of the nanochelators from the hydrogel would have a positive effect on the treatment of iron overload. Interestingly, F127 micelles tend to be absorbed by the skin near the injection site, which might explain the high fluorescence signals on the skin in the DFO-NP/F127 and DFO-NP/HA/F127 treatment groups (FIG. 7a). In addition to higher skin SBR, the SBR of the remaining hydrogel in the DFO-NP/HA/F127 group was higher than others suggesting that DFO-NPs can be released when the hydrogel decomposes. Moreover, we observed the biodistribution of DFO-NP in all experimental groups after 14 days post-injection (FIG. 8). In all groups, no fluorescence signal was observed in organs other than the kidney and bladder which clearly showed that our nanochelator, DFO-NP, has exclusive urinary excretion and no off-target potential toxicity.

### Pharmacokinetics Study

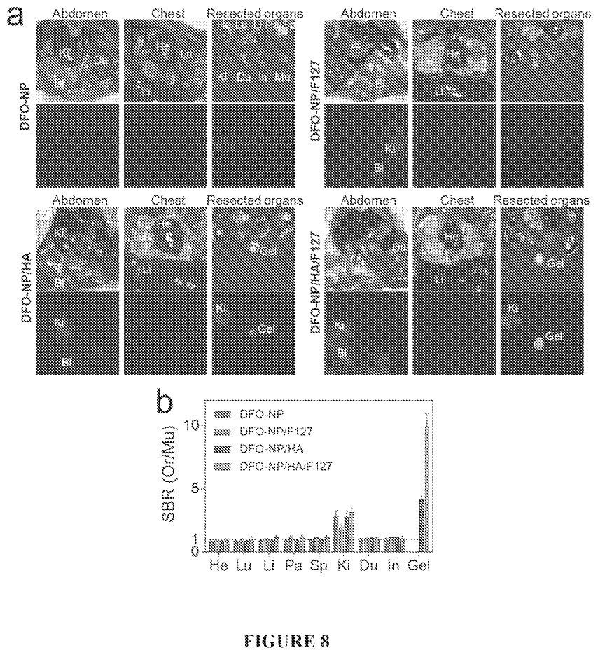

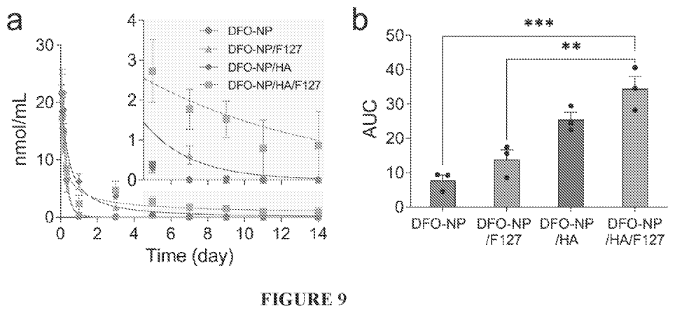

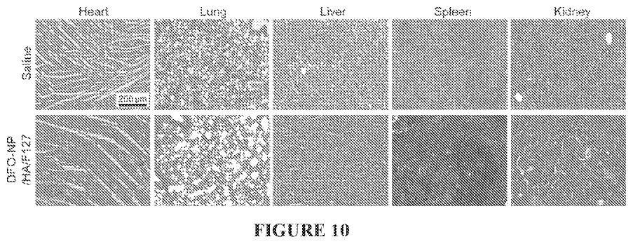

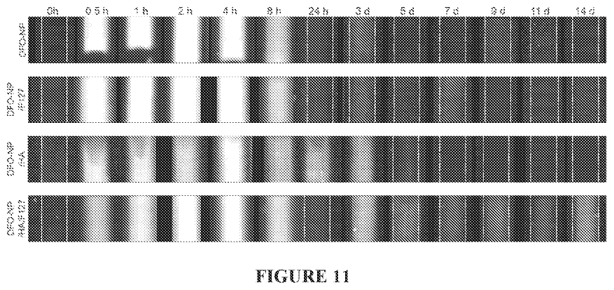

To further investigate the sustained release of DFO-NP from injectable hydrogel formulations, a PK study was performed. Mouse blood samples were collected at predetermined time points throughout 14 days, and the concentration of DFO-NP in the blood was determined at each time point by NIR fluorescent signal intensity, which was used to produce plasma concentration decay curves of the injected formulations (FIG. 9a and FIG. 10).

The plasma concentrations of DFO-NP in DFO-NP/F127 and DFO-NP groups were barely detectable after 3 days. This result indicates that Pluronic F127 alone is not suitable for the sustained release of DFO-NP and that DFO-NP absorbed into the skin with Pluronic F127 micelle is hardly released. The concentration in the DFO-NP/HA group was well maintained until day 3. However, almost no fluorescence signal from DFO-NP (<0.05 nmol ml-1) was observed after 5 days post-injection. In the DFO-NP/HA/F127 group, the concentration of DFO-NP exhibited a similar pattern to that of the DFO-NP/F127 group which is mainly due to the presence of Pluronic F127 micelles in both groups. In contrast to formerly stated formulations, the signal intensity gradually decreased until 14 days but was maintained at 1 nmol ml-1 which is similar to the level of DFO-NP alone when intravenously injected (2 μmol kg-1) at 1 h post-injection. This concentration decay suggests that DFO-NP/HA/F127 has a sustained-release property. For further comparison, PK parameters of the formulations were calculated from the decay curve. The area under the curve (AUC) of DFO-NP/HA/F127 is the highest among the different formulations and is 4-fold higher than that of DFO-NP alone (***p<0.001 compared to DFO-NP and **p<0.005 compared to DFO-NP/F127 groups) as shown in FIG. 9b. All calculated PK parameters (half-life, bioavailability, Cₘₐₓ, Tₘₐₓ, and K_{slow}) exhibited that the PK of DFO-NP was significantly improved when formulated with the HA/F127 hydrogel (Table 1). In particular, the half-life of DFO-NP/HA/F127 is 47, 35, and 4-fold longer and the bioavailability is 4, 2.5, and 1.3-fold higher compared to DFO-NP, DFO-NP/HA, and DFO-NP/F127, respectively. From these results, it was confirmed that DFO-NP/HA/F127 successfully delayed the release of DFO-NP.

TABLE 1
pharmacokinetic parameters of each sample.

| | Half-Life | Bioavailability^{†} | | | |
|---|---|---|---|---|---|
| Group | (d) | (%) | Cₘₐₓ | Tₘₐₓ | K_{slow} |
| DFO-NP | 0.15 | — | 32.27 | 0.083 | 4.7 |
| DFO-NP/F127 | 0.2 | 179 ± 60 | 28.91 | 0.083 | 3.5 |
| DFO-NP/HA | 1.8 | 329 ± 46 | 24.96 | 0.1667 | 0.4 |
| DFO-NP/HA/F127 | 7.0 | 444 ± 80 | 23.59 | 0.083 | 0.1 |

^{†}Relative bioavailability (AUC_{Hydrogel}/AUC_{DFO-NP} × 100) compared to DFO-NP.
The concentrations of DFO-NP in plasma were calculated by measuring fluorescence signal intensity (n = 3 per group, mean ± s.e.m.).
p values < 0.05 were considered significant: ** p < 0.01, *** p < 0.005

### Example 5: In Vivo Toxicity Test of DFO-NP Loaded Injectable Hydrogel Formulation

Toxicity study: To evaluate the toxicity of DFO-NP and DFO-NP loaded hydrogel formulations, blood samples were collected by cardiac puncture 14 d after injection. These blood samples were stored at room temperature without anticoagulant for 30 min and were then centrifuged at 3,000 rpm for 15 min. Serum was stored at −80° C. until further assays. We measured activities of aspartate aminotransferase (AST) and alanine aminotransferase (ALT) for hepatotoxicity, and blood urea nitrogen (BUN) and creatinine (CREA) for nephrotoxicity. All assays were performed using commercially available assay kits and the absorbance was immediately measured by a plate reader.

Statistical analysis: The fluorescence and background intensities of a region of interest over each organ were quantified using customized imaging software and ImageJ v1.52i (National Institutes of Health, Bethesda, MD). The signal-to-background ratio (SBR) was calculated as SBR=fluorescence/background, where the background is the fluorescence intensity of muscle or system background. Data are presented as mean±s.e.m. with a minimum of three replicates. Student's t-test statistical analysis was performed to evaluate the significance of the experimental data. The differences among groups were determined using one-way ANOVA analysis with Bonferroni's multiple comparison corrections to assess the statistical differences among more than two groups. p values of less than 0.05 were considered significant. The data was indicated with *p<0.05, **p<0.01, and ***p<0.001.

The chronic toxicity of DFO-NP was evaluated when DFO-NP was formulated with DFO-NP/HA/F127 at 30/7/30% and injected subcutaneously. For histological analysis, hematoxylin and eosin (H&E) staining was performed for major organ samples (heart, lung, liver, spleen, and kidney) acquired on 14 days post-injection. (FIG. 9a and FIG. 10). In the H&E staining images of all organs, no pathological differences were observed between the saline and DFO-NP/HA/F127 injection groups, even in the kidneys which are the most exposed organ to DFO-NP.

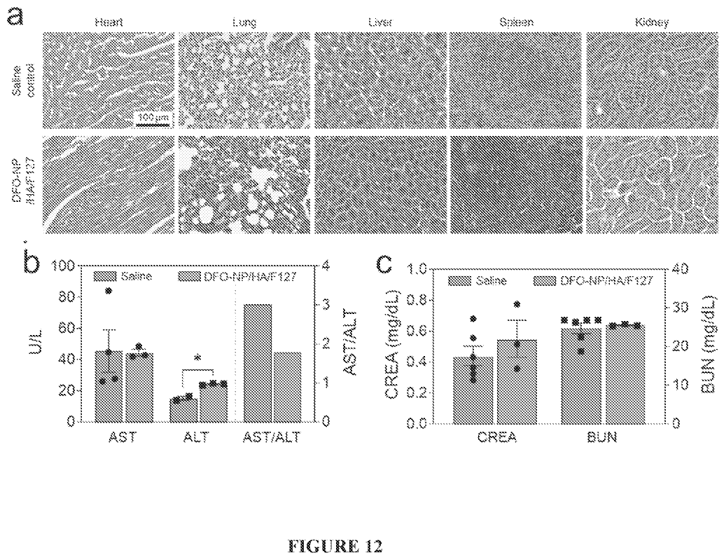

For further quantitative assessment for hepatotoxicity and nephrotoxicity, biochemical analyses of aspartate aminotransferase (AST), alanine transaminase (ALT), blood urea nitrogen (BUN), and creatinine (CREA) were performed. As shown in FIG. 12b, there was no difference in AST levels between the saline and DFO-NP/HA/F127 groups. ALT levels of DFO-NP/HA/F127 were slightly higher than that of the saline control group (*p<0.05), but within the normal range for mice (25-60 U/L^{[24]}). This suggests that DFO-NP/HA/F127 does not induce hepatotoxicity. In addition, there was no significant difference in CREA and BUN levels between the saline and DFO-NP/HA/F127 groups (FIG. 12c). This indicates that a high dose of the DFO-NP/HA/F127 formulation does not induce any chronic toxicity.

### Example 6: In Vivo Therapeutic Efficacy of DFO-NP Loaded Injectable Hydrogel Formulation

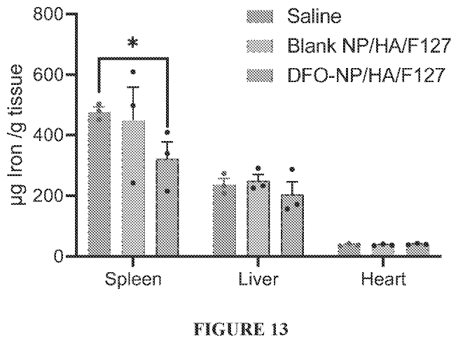

To confirm the in vivo therapeutic efficacy of DFO-NP formulations, DFO-NP/HA/F127 hydrogel was subcutaneously injected into dietary induced iron overload (DIO) mice. For comparisons, an empty hydrogel, i.e., nanochelators without DFO (blank NP) including the same hydrogel formulation, was injected subcutaneously. Saline was used as a control. Organs (spleen, liver, and heart) were collected 3 weeks post-administration of each sample, and the iron content in the organs was measured quantitatively using ICP-MS (FIG. 13). We confirmed a significant difference in iron levels in the spleen between the DFO-NP/HA/F127 and saline groups (Δ156.9 μg iron/g tissue: p<0.05 compared to saline control group) and a tendency of lower iron content in the liver (Δ33.0 μg iron/g tissue). The higher efficacy of iron excretion in the spleen than in the liver is likely due to greater iron overload in the spleen than in the liver.

### Other Embodiments

Whilst the application has been disclosed in particular embodiments, it will be understood by those skilled in the art that certain substitutions, alterations and/or omissions may be made to the embodiments without departing from the spirit of the invention. Accordingly, the foregoing description is meant to be exemplary only, and should not limit the scope of the invention. All references, scientific articles, patent publications, and any other documents cited herein are hereby incorporated by reference in their entireties.

1. A sustained release, injectable composition comprising an iron chelation agent and at least one hydrogel.

2. The composition of claim 1, wherein the composition is capable of release of the iron chelation agent from about 1 day to about 30 days.

3. The composition of claim 2, wherein the composition releases the iron chelation agent over a period of at least two weeks.

4. The composition of any one of claims 1-3, wherein the hydrogel comprises hyaluronic acid (HA).

5. The composition of claim 3, wherein the HA is crosslinked.

6. The composition of claim 4, wherein the HA is crosslinked with 1,4 butanediol diglycidyl ether (BDDE), 1,2-ethanediol diglycidyl ether (EDDE), ethylene glycol diglycidyl ether (EGDE), 1,2-bis(2,3-epoxypropoxy)ethylene (EGDGE), or diepoxyoctane.

7. The composition of claim 1, wherein the hydrogel further comprises F127.

8. The composition of claim 7, wherein the composition comprises 30 wt % F127, 7 wt % HA, and 30 wt % iron chelation agent.

9. The composition of claim 1, wherein the iron chelation agent is defereoxamine (DFO).

10. The composition of claim 9, wherein the DFO is DFO nanochelator (DFO-NP).

11. A method of treating iron overload in a patient in need thereof comprising administering a therapeutically effective amount of a composition of claim 1 to the patient.

12. A method of chelating a metal ion in a cell or tissue sample, comprising contacting a cell sample or a tissue sample with a composition of claim 1.

13. A method of reducing the amount of free metal ions in a cell or tissue sample, comprising contacting a cell sample or a tissue sample with a composition of claim 1.

14. A method of treating a disease associated with an abnormal amount of free metal ions in a subject, comprising administering to a subject determined to have an abnormal level of free metal ions a composition of claim 1.

15. The method of claim 14, wherein the disease is associated with an abnormal amount of iron ions, an abnormal amount of lead ions, or an abnormal amount of copper ions in the subject, or any combination thereof.

16. The method of claim 14, wherein the disease is associated with an abnormal amount of iron ions in the subject.

17. The method of claim 14, wherein the disease is selected from transfusion hemosiderosis, hemochromatosis, Wilson's disease, copper poisoning, and heavy metal poisoning.

18. A method of preparing a sustained-release, injectable composition useful iron chelation therapy comprising the steps:
a) cross-linking HA;
b) making the cross-linked HA into particles;
c) adding the particles of cross-linked HA into a solution of DFO-NP; and
d) further adding F127 to the resulting solution of step c).

19. A sustained release, injectable composition for iron chelation therapy in a human patient comprising an iron chelation agent and a hydrogel, said composition capable of release over a two week period.

20. The composition of claim 13 in which the hydrogel is composed of HA and F127.

21. The composition of claim 14 in which the HA is crosslinked.

22. The composition of claim 15 in which the HA is crosslinked with BDDE.

23. The composition of claim 14 in which the weight percent of the components is 30 wt % F127, 7 wt % HA, and 30 wt % DFO-NP.

24. The composition of claim 14 in which the iron chelation agent is DFO.

25. The composition of claim 18 in which the DFO is DFO-NP.

26. A method of treating iron overload in a patient comprising administering an effective amount of a composition of claim 13 to the patient.

27. Any and all compositions, articles of manufacture, methods, and uses disclosed and/or described herein.
